(19) United States
(12) Patent Application Publication
(10) Pub. No.: US 2002/0156486 A1
(43) Pub. Date: Oct. 24, 2002

(54) Optical lens furler and injector

(76) Inventor: Bernard A. Nadel, Irvington, VA (US)

Correspondence Address: BERNARD A NADEL, 49 REYNOLD'S LANDING, P.O. Box 499, IRVINGTON, VA, 22480, US

(21) Appl. No.: 10/081,846

(22) Filed: May 9, 2002

Related U.S. Application Data
(60) Provisional application No. 60/284,808, filed on Apr. 19, 2001.

(51) Int. Cl. A61F 9/00

(52) U.S. Cl. 606/107

(57) ABSTRACT

In ophthalmology a cataracted lens is removed from the eye and replaced with an artificial one. Presently, an artificial lens, packaged in a clean room is shipped in a sterile package. The surgeon, after excising the cataracted lens, removes the artificial lens from the package and inserts it into the eye. The success of the operation done in this manner is subject to the skill of the surgeon, and possible contamination. A large incision requiring stitching is necessary unless the lens is folded. Folding tends to exceed the modulus of elasticity of the lens and a lens folded in this manner unfolds uncontrollably in the eye and needs adjustment.

The Furler & Injector, which curls or furls the lens, is also the package and the lens is never exposed to the air. A curled or furled lens uncurls gradually and stays put. Further, using elements of this invention, the entire operation including excision may be done mechanically independent of the surgeon's dexterity and not one stitch is required.

### RELATED APPLICATION

The benefit of the filing date of provisional application number 60/284,808, filing date Apr. 19, 2001 entitled Optical Lens Furler and Injector is hereby claimed.

### BACKGROUND

1. Field of Invention

The present invention relates to the methods of and apparatus for preparing and inserting flexible intra ocular lenses used in the field of ophthalmology.

2. Description of Prior Art

In cataract surgery, a cataractous human lens is removed through a 3 mm or larger incision. A prosthetic intra ocular lens is then substituted for the human lens. The intra ocular lens' transparency improves the patient's or restores the patient's vision. In practice, a smaller incision is necessary if the prosthetic lens is folded prior to insertion. obviating the need for additional vision correction after surgery, which would otherwise be necessary.

Intra ocular lenses may be made of flexible materials such as silicone. Although lenses made of these materials have dimensions in their uncompressed state which are larger than 3 mm, such lenses may be folded in various configurations and inserted through 3 mm or slightly larger incisions. When using flexible intra ocular lenses, surgical incisions necessary for performing cataract surgery need not be enlarged.

Flexible intra ocular lenses have two essential components, the first being a central optic component which is round or oval in shape. They are between five and seven millimeters in diameter. The optic component replaces the cataractous lens after cataract extraction. The second component(s) are attached to the optic component, and extend peripherally therefrom. Known as haptics, these provide internal fixation and centration of the intra ocular lens after its insertion into the eye. The haptics may be flexible plates extending outward from the optic component as a unitary extension thereof. Alternatively, the haptics may be joined to the optic component and configured as open loops, termed “C” or “J” loops. The function of both haptic structures is similar.

U.S. Pat. No. 5,556,400 by Scott W. Tunis contains an excellent background description which is incorporate herein by reference and some of which is also incorporate herein by inclusion.

The Tunis invention however has shortcomings which are overcome by the present invention. Use of the Tunnis invention requires several folds in the intra ocular lens through the use of forceps which requires exceptional dexterity on the part of the surgeon and also exposes the lens to damage both through the use of multiple folds which may exceed it's modulus of elasticity and by the forceps used to fold the lens. Furthermore, If damage is imparted to the lens it is hidden from the surgeons view until after the lens is implanted in the eye. The Tunis invention further exposes the lens to possible contamination in the folding process.

In an unrelated art, sewing machine accessory devices for folding fabric are well known. They are mentioned here in the interest of full disclosure though the materials used and the art area are totally unrelated to ophthalmology.

### OBJECTS AND ADVANTAGES OF THE CURRENT INVENTION

Accordingly several objects and advantages of the current invention are:

(a) to provide a sterile carrier in the form of a syringe for the intra ocular lens such that the lens is not exposed to any environmental contamination from the time it is packaged in a factory clean room to the time it is inserted in the patient's eye, The syringe is the package and obviates the need for the complicated and expensive sterile package now in use.

(b) to provide accommodation for visual inspection of the lens in the carrier,

(c) to provide a tool for furling the lens to a diameter which is significantly smaller than its unfurled diameter,

(d) to provide a tool which can furl the lens with minimum dexterity in a clean room and subsequently aid the surgeon to immobilize and insert the lens into the eye.

(e) to provide an optical positioner to aid the physician in determining the location for an incision in the eye by marking the eyeball.

(f) to provide an alignment mechanism to position a scalpel to follow a path as determined by the optical positioner and also to position the furling tool for insertion of the lens into the eye.

(g) to provide a rotating scalpel to partially complete a circular incision, immobilize the eye, enter the eye, macerate the cataractous lens and suck out the macerated particles.

(l) To provide an indexing device for juxtapositioning the rotating scalpel and the furler to follow a path preset by the optical positioner.

### SUMMARY OF INVENTION

The present invention is both a method for using and a intra ocular lens tool comprised of a syringe like instrument in which the intra ocular lens is transported in a first portion which is generally rectangular in cross section. In use the lens is urged by use of a plunger through a second portion transitioning from generally rectangular to a conical, overlapped section, and then into a tubular portion. The lens is inserted into the eye by further movement of the plunger to eject the lens through the eye incision. An indexable turret device may be used to juxtaposition the paths for the necessary surgical operations. as follows: 1. An optical positioner is used to predetermine and mark the ideal path. 2 The turret is indexed. A rotating scalpel is deployed. An incision is made. The eye is entered and immobilized. The lens is macerated and sucked out. 3. The turret is indexed. The Furler and Injector is deployed. The new lens is inserted and affixed into the eye.

Upon further study of the specification and appended claims, further objects and advantages of this invention will become apparent to those skilled in the art.

### DESCRIPTION OF DRAWINGS

Various other objects, features and attendant advantages of the present invention will be more fully appreciated as the same becomes better understood when considered in conjunction with the accompanying drawings.

Sheet 1 of 5

FIG. 1 Illustrates an intra ocular lens 10 with optic component 20 and haptics 30.

FIG. 1A, Inset illustrates Furled Lens Enlarged.

FIG. 2a, 2b and 2c illustrate the syringe assembly 40 with the plunger 50 inserted into the syringe shell 60.

FIG. 3 shows in detail syringe plunger 50.

FIG. 4 shows the syringe shell 60 which is made from transparent material.

Sheet 2 of 5

FIG. 5 shows the syringe assembly 40 being used to insert a lens into the eye of a patient. The entire furling transition can be visually monitored.

Sheet 3 of 5

FIG. 6 shows an optical positioner 61,

FIG. 6A (inset) shows tip enlarged.

FIG. 7 shows a rotating scalpel 81,

FIG. 7A (inset) shows tip enlarged.

Sheet 4 of 5

FIG. 8 illustrates adjustable head brace 84 with immobilizing nut 82 holding optical positioner 61 in juxtaposition for alignment with lens.

FIG. 9 is similar to FIG. 8 but holds the rotating scalpel 81 in juxtaposition predetermined in FIG. 8. 101 is a vacuum pump attached to the scalpel by a flexible hose 102

FIG. 10 is similar to FIGS. 8 and 9 but now holds the Lens Furler and Injector.40 in juxtaposition for lens insertion. All juxtapositions are indexed via a turret not shown for clarity. See Sheet 5

Sheet 5 of 5

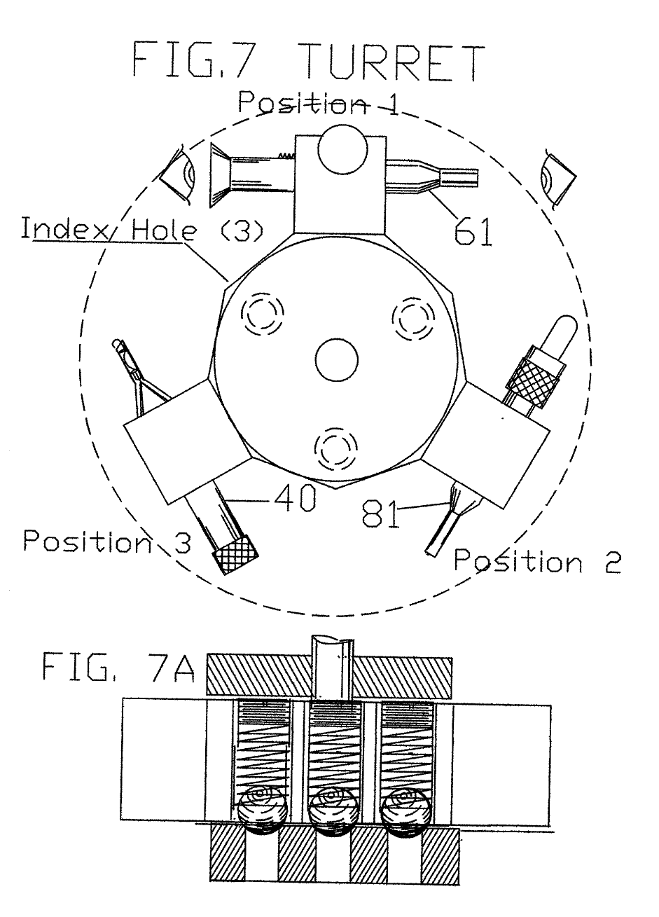

FIG. 7 illustrates an indexing turret with cross section 7A illustrating optical positioner 61 deployed in position 1 to be followed by rotating scalpel 81 indexed to position 2.and by the Lens Furler and Injector indexed to final position 3.

### DETAILED DESCRIPTION

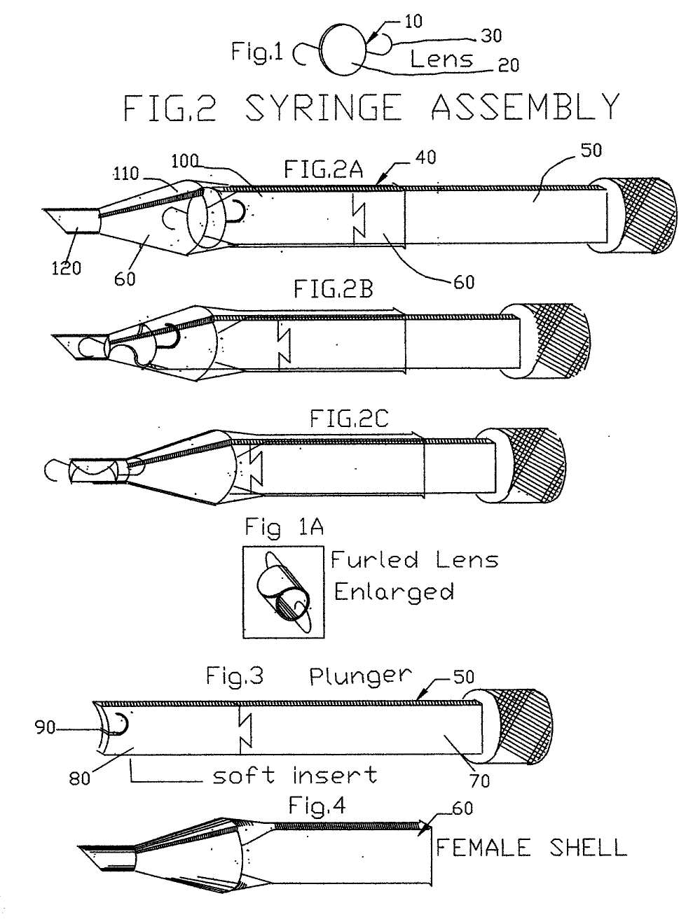

The present invention provides methods of and an intra ocular lens tool for transporting, furling, and inserting flexible intra ocular lenses into the eye with a mechanism to accurately position the lens tool after the mechanism has been aligned through the use of an optical positioner which locates and marks by pigmentation or penetration producing a semi circular mark or incision. The mark or incision is then located and completed through the use of a rotating scalpel. Referring to FIG. 3, Sheet 1, at the factory where the lens is manufactured, under sterile conditions the plunger 40 is assembled with rigid portion 70 and soft flexible portion 80. Lens of FIG. 1 is placed at the end of the soft flexible portion 80 with one of the haptics 30 cooperating with the groove 90 in the flexible portion 80. This plunger assembly is then inserted into the syringe shell 60 and advanced to position 1, FIG. 2A, A lubricant (not shown) is used during the assembly process to facilitate the operation of the device as well as maintain sterile conditions in the syringe assembly. The entire assembly 40 is then packaged in sterile packaging for transport to the physician.

In the operating room the Lens Furler and Injector 40 is removed from the sterile packaging after the patient's eye has been prepared to receive the lens. The plunger 50, as illustrated in FIGS. 2A and 2B is pushed into the shell portion 60. FIG. 2B. The lens and tip 80 are being furled up as they make a transition from the rectangular portion 100 into the conical portion 110 and on into the tubular portion 120.The conical transition portion has a diminishing radius so that it can furl (overlap) to produce an even smaller diameter than if it were merely rolled. See inset FIG. 1A enlarged.

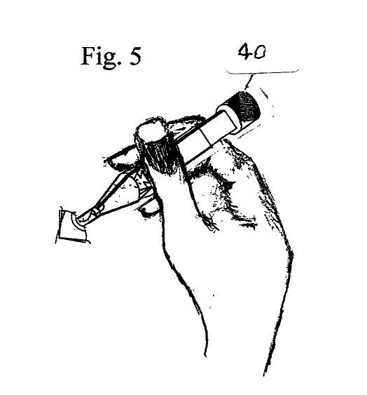

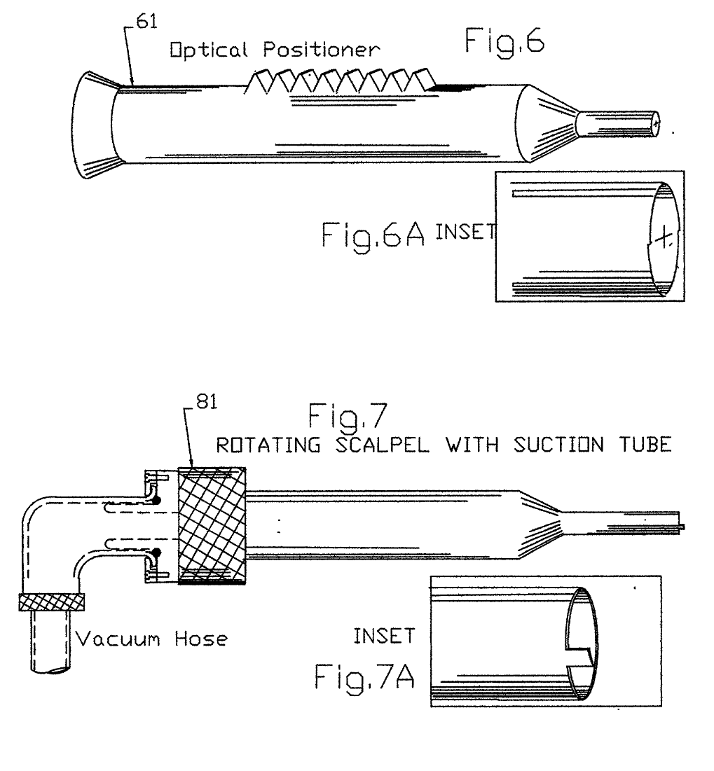

When the lens reaches the outer extremity of the shelf portion as illustrated in FIG. 2c, it shall have already been inserted into the now immobilized eye as in FIG. 5, Sheet 2.

The procedure when the physician elects to use a mechanical means for positioning the instruments is as follows:

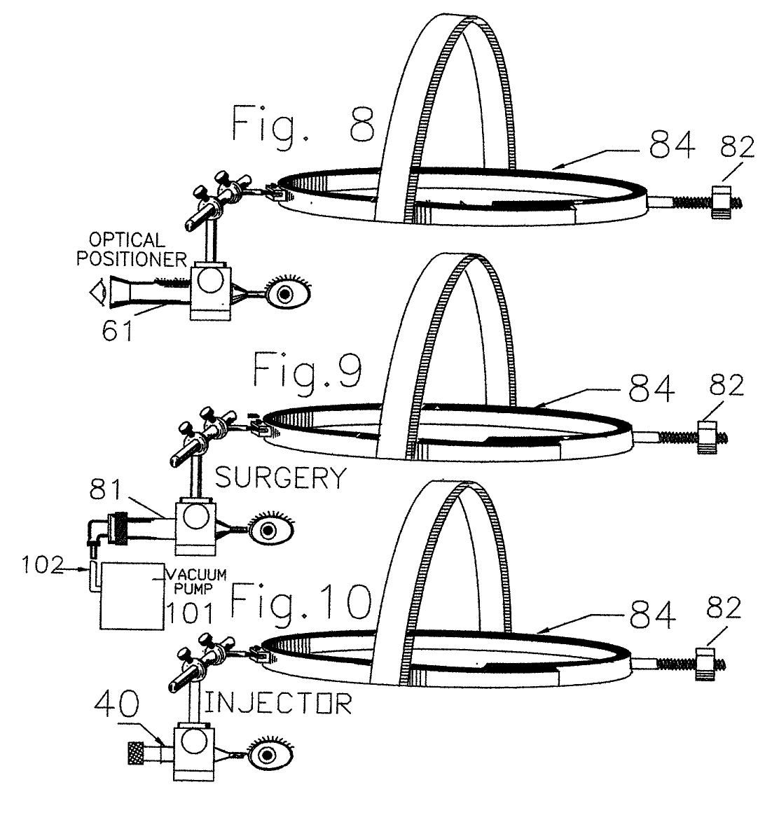

The patient is prepared. The head brace FIGS. 8,9 and 10, sheet 4 is finally attached with hook and loop fastening straps and immobilized further by attaching the brace to a chair via the screw 82.

The sterilized optical positioner 61, a microscope with cross hairs, has been deployed into the head brace as shown in FIG. 8. Patient not shown. The surgeon locates a perfect path (juxtaposition) by aligning the Optical Positioner's cross hairs, and marks a semi circle with pigment or advances the positioner to produce a semi circular incision.

In position 2, FIG. 9, the sterilized Rotating Scalpel 81, which has also been deployed, is indexed to locate the pigmentation or incision made by the Optical Positioner.61.

The surgeon completes the circular incision by rotating the scalpel to a point less than 360 degrees, leaving a flap. He then enters the eye which is now immobilized by the tubular portion of the scalpel and macerates the diseased lens by rotation and sucks out the remains using a foot operated vacuum pump sump 101. The Lens Furler and Injector 40, which has been unpacked and deployed into the head brace, is now indexed to align with the incision for final position FIG. 10. The surgeon enters the eye with the tubular section. The eye again is immobilized. The plunger is depressed and the new lens is injected and located in the eye.

The lens gradually and gently resumes its normal geometry and therefore cannot reenter the syringe and perforce remains precisely where placed when the plunger is easily withdrawn and the lens and haptics require minimum if any post manipulation.

The efficiency of the surgical improvements illustrated, especially the gentle release which solves a heretofore difficulty should not only assure more successful results but in addition should shorten the time for these results to take place thereby decreasing the discomfort of the patient. In the future the entire procedure from beginning to end could be automated and done remotely.

### CONCLUSIONS, RAMIFICATIONS, AND SCOPE

As the reader can see, the inventor has devised an apparatus and method for transporting, furling, and inserting a lens into the eye of a cataract patient including a method for removal of the cataracted lens. Additional advantages and embodiments will become apparent through the use of the invention and thus the scope of the invention should be determined by the appended claims and their legal equivalents rather than by the examples given.

1. A device used in Ophthalmology that sterilely delivers a cataract replacement lens from a clean room to the surgeon, where the Lens Furler and Ejector is at once, the delivery package; the curler or furler of the replacement lens that affords full visual inspection of the curling or furling as it occurs and the means of injection directly into the eye wherein the lens, from the clean room to the required position within the eye is never exposed to the atmosphere, thus precluding the possibility of contamination.

2. A device that makes a precise tiny circular incision on the eyeball; enters and immobilizes the eye and macerates and sucks out the diseased lens.

3. An indexable device that, in conjunction with the Lens Furler and Ejector, optically locates and coordinates devices in claims 1 and 2 in juxtaposition to assist the surgeon to more successfully and more quickly, complete a lens transplant operation.

4. The elements of a robotic machine comprised of the devices in claims 1, 2, and 3, that under supervision or remote control, could automatically perform a cataract transplant better, faster and with greater precision than could be done manually.
